(19) United States
(12) Patent Application Publication
(10) Pub. No.: US 2024/0149499 A1
(43) Pub. Date: May 9, 2024

(54) MOLD MANUFACTURING SYSTEM

(71) Applicant: Zhong Chuan Technology Limited, Taoyuan City (TW)

(72) Inventor: YU-PAU LIN, Taoyuan City (TW)

(21) Appl. No.: 18/107,561

(22) Filed: Feb. 9, 2023

(30) Foreign Application Priority Data
Nov. 7, 2022 (TW) 111142317

(51) Int. Cl. B29C 33/38; B22C 11/02

(52) U.S. Cl. CPC B29C 33/3842; B22C 11/02

(57) ABSTRACT

A mold manufacturing system includes a central control module, a robot arm module, a mold placement module, a tool placement module, a material processing module and a mold cleaning module. The robot arm module is movably disposed on a system track. The mold placement module includes a mold carrying frame configured to carry a plurality of mold materials that have not been processed and a plurality of semi-finished molds that have been processed. The tool placement module includes a tool carrying frame configured to carry a plurality of processing tools. Each of the processing tools includes a tool holder and a tool detachably clamped by the tool holder. The material processing module includes a plurality of material processing machines. The mold cleaning module includes a first cleaning tank for accommodating liquid medicine, a second cleaning tank for accommodating clear water and a drying tank for providing hot air.

### CROSS-REFERENCE TO RELATED PATENT APPLICATION

This application claims the benefit of priority to Taiwan Patent Application No. 111142317, filed on Nov. 7, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a mold manufacturing system, and more particularly to an automated mold manufacturing system.

### BACKGROUND OF THE DISCLOSURE

In a modern industrial production process, although machines and factories have replaced humans and animals as primary sources for productivity, manpower is still used for transporting materials and molds. As a result, the efficiency of the production process cannot be optimized, and labor costs cannot be reduced.

### SUMMARY OF THE DISCLOSURE

In response to the above-referenced technical inadequacy, the present disclosure provides a mold manufacturing system that is capable of making molds in an automated manner.

In order to solve the above-mentioned problems, one of the technical aspects adopted by the present disclosure is to provide a mold manufacturing system, which includes a central control module, a robot arm module, a mold placement module, a tool placement module, a material processing module, a mold cleaning module, a dimension measurement module and an electrical discharge machining module. The robot arm module is electrically connected to the central control module, and the robot arm module is movably disposed on a system track. The mold placement module is electrically connected to the central control module, the mold placement module includes a mold carrying frame and a first movable safety door adjacent to the mold carrying frame, and the mold carrying frame is configured to carry a plurality of mold materials that have not yet been processed and a plurality of semi-finished molds that have been processed. The tool placement module is electrically connected to the central control module, the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder. The material processing module is electrically connected to the central control module, the material processing module includes a plurality of material processing machines, and each of the material processing machines is configured to use one of the processing tools to process one of the mold materials, thereby obtaining a corresponding one of the semi-finished molds. The mold cleaning module is electrically connected to the central control module, and the mold cleaning module includes a first cleaning tank for accommodating liquid medicine, a second cleaning tank for accommodating clear water and a drying tank for providing hot air. The dimension measurement module is electrically connected to the central control module, and the dimension measurement module is configured to measure the semi-finished mold, thereby obtaining actual dimension information of the semi-finished mold. The electrical discharge machining module is electrically connected to the central control module, and the electrical discharge machining module is configured to perform precision machining on the semi-finished mold according to a difference between the actual dimension information and standard dimension information, thereby making the difference between the actual dimension information of the semi-finished mold and the standard dimension information is within a predetermined error range. The mold placement module, the tool placement module, the material processing module, the mold cleaning module, the dimension measurement module and the electrical discharge machining module are all adjacent to the system track and arranged along the system track. The robot arm module is configured to be movably disposed among the mold placement module, the tool placement module, the material processing module, the mold cleaning module, the dimension measurement module and the electrical discharge machining module and along the system track.

In order to solve the above-mentioned problems, another one of the technical aspects adopted by the present disclosure is to provide a mold manufacturing system, which includes a central control module, a robot arm module, a mold placement module, a tool placement module, a material processing module, a mold cleaning module and a dimension measurement module. The robot arm module is electrically connected to the central control module, and the robot arm module is movably disposed on a system track. The mold placement module is electrically connected to the central control module, the mold placement module includes a mold carrying frame and a first movable safety door adjacent to the mold carrying frame, and the mold carrying frame is configured to carry a plurality of mold materials that have not yet been processed and a plurality of semi-finished molds that have been processed. The tool placement module is electrically connected to the central control module, the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder. The material processing module is electrically connected to the central control module, the material processing module includes a plurality of material processing machines, and each of the material processing machines is configured to use one of the processing tools to process one of the mold materials, thereby obtaining a corresponding one of the semi-finished molds. The mold cleaning module is electrically connected to the central control module, and the mold cleaning module includes a first cleaning tank for accommodating liquid medicine, a second cleaning tank for accommodating clear water and a drying tank for providing hot air. The dimension measurement module is electrically connected to the central control module, and the dimension measurement module is configured to measure the semi-finished mold, thereby obtaining actual dimension information of the semi-finished mold. The mold placement module, the tool placement module, the material processing module, the mold cleaning module and the dimension measurement module are all adjacent to the system track and arranged along the system track. The robot arm module is configured to be movably disposed among the mold placement module, the tool placement module, the material processing module, the mold cleaning module and the dimension measurement module and along the system track.

In order to solve the above-mentioned problems, yet another one of the technical aspects adopted by the present disclosure is to provide a mold manufacturing system, which includes a central control module, a robot arm module, a mold placement module, a tool placement module, a material processing module and a mold cleaning module. The robot arm module is electrically connected to the central control module, and the robot arm module is movably disposed on a system track. The mold placement module is electrically connected to the central control module, the mold placement module includes a mold carrying frame and a first movable safety door adjacent to the mold carrying frame, and the mold carrying frame is configured to carry a plurality of mold materials that have not yet been processed and a plurality of semi-finished molds that have been processed. The tool placement module is electrically connected to the central control module, the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder. The material processing module is electrically connected to the central control module, the material processing module includes a plurality of material processing machines, and each of the material processing machines is configured to use one of the processing tools to process one of the mold materials, thereby obtaining a corresponding one of the semi-finished molds. The mold cleaning module is electrically connected to the central control module, and the mold cleaning module includes a first cleaning tank for accommodating liquid medicine, a second cleaning tank for accommodating clear water and a drying tank for providing hot air. The mold placement module, the tool placement module, the material processing module and the mold cleaning module are all adjacent to the system track and arranged along the system track. The robot arm module is configured to be movably disposed among the mold placement module, the tool placement module, the material processing module and the mold cleaning module and along the system track.

In one of the possible or preferred embodiments, the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader.

In one of the possible or preferred embodiments, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base.

In one of the possible or preferred embodiments, the mold placement module is configured to provide mold material information and semi-finished mold information to the central control module, the mold material information includes mold material quantity information and mold material location information, and the semi-finished mold information includes semi-finished mold quantity information and semi-finished mold location information.

In one of the possible or preferred embodiments, the mold material quantity information and the mold material location information of the mold material information and the semi-finished mold quantity information and the semi-finished mold location information of the semi-finished mold information are all displayed on an information display electrically connected to the central control module, and the mold material quantity information and the mold material location information of the mold material information and the semi-finished mold quantity information and the semi-finished mold location information of the semi-finished mold information are transmitted to a portable electronic device of a user through the central control module.

In one of the possible or preferred embodiments, the mold placement module includes a first sensor adjacent to the first movable safety door, and the first sensor is configured to determine whether the first movable safety door is in an open state or a closed state.

In one of the possible or preferred embodiments, the tool placement module includes a second sensor adjacent to the second movable safety door, and the second sensor is configured to determine whether the second movable safety door is in an opened state or a closed state.

In one of the possible or preferred embodiments, the mold materials and the semi-finished molds are respectively and detachably disposed on a plurality of corresponding mold carrying seats, and each of the mold carrying seats has a mold identification barcode that is configured to be read by the barcode reader of the robot arm, and mold information provided by the mold identification barcode of the mold carrying seat corresponds to a type of the mold material or a type of the semi-finished mold that is carried by the mold carrying seat.

In one of the possible or preferred embodiments, the tool holder has a tool identification barcode that is configured to be read by the barcode reader of the robot arm, and tool information provided by the tool identification barcode of the tool holder corresponds to a type of the tool clamped by the tool holder.

In one of the possible or preferred embodiments, the tool placement module further includes a rotatable carrier for carrying the tool holder clamping the tool, and a tool holder barcode reader adjacent to the rotatable carrier.

In one of the possible or preferred embodiments, when the tool holder clamping the tool is rotated by rotating of the rotatable carrier so as to rotate the tool identification barcode of the tool holder to face the tool holder barcode reader, the tool holder barcode reader is configured to read the tool identification barcode of the tool holder.

In one of the possible or preferred embodiments, when the robot arm of the robot arm module is configured to use the at least one mold gripping jaw, the mold carrying seat carrying the semi-finished mold is immersed into the first cleaning tank containing the liquid medicine by clamping of the at least one mold gripping jaw, so that the semi-finished mold is cleaned by the liquid medicine through ultrasonic vibration.

In one of the possible or preferred embodiments, when the robot arm of the robot arm module is configured to use the at least one mold gripping jaw, the mold carrying seat carrying the semi-finished mold is transferred from the first cleaning tank to the second cleaning tank and immersed into the second cleaning tank containing the clear water by clamping of the at least one mold gripping jaw, so that the semi-finished mold is cleaned by the clear water through ultrasonic vibration.

In one of the possible or preferred embodiments, when the robot arm of the robot arm module is configured to use the at least one mold gripping jaw, the mold carrying seat carrying the semi-finished mold is transferred from the second cleaning tank to the drying tank by clamping of the at least one mold gripping jaw, so that the semi-finished mold is dried by the hot air provided by the drying tank so as to remove excess moisture.

In one of the possible or preferred embodiments, the mold cleaning module further includes a first heater configured to provide heat source for the first cleaning tank, a second heater configured to provide heat source for the second cleaning tank, and a hot air provider configured to provide heat source for the drying tank.

In one of the possible or preferred embodiments, the material processing module includes a plurality of barcode confirmation sensors respectively disposed inside the material processing machines, and each of the barcode confirmation sensors is configured to read the mold identification barcode of the mold carrying seat and the tool identification barcode of the tool holder in the material processing machine, thereby confirming whether or not the mold material and the tool used in the material processing machine is correct.

In one of the possible or preferred embodiments, the mold cleaning module further includes a first water quality detector disposed in the first cleaning tank and a second water quality detector disposed in the second cleaning tank, the first water quality detector is configured to detect a water quality of the liquid medicine in the first cleaning tank, and the second water quality detector is configured to detect a water quality of the clear water in the second cleaning tank.

In one of the possible or preferred embodiments, the dimension measurement module includes an image dimension measuring instrument, a surface 3D profile measuring instrument or a coordinate measuring machine.

In one of the possible or preferred embodiments, after the actual dimension information of the semi-finished mold is transmitted from the dimension measurement module to the central control module, the electrical discharge machining module is controlled by the central control module for precisely processing the semi-finished mold according to the actual dimension information.

Therefore, in the mold manufacturing system provided by the present disclosure, by virtue of “the robot arm module being movably disposed on a system track,” “the mold placement module including a mold carrying frame and a first movable safety door adjacent to the mold carrying frame, and the mold carrying frame being configured to carry a plurality of mold materials that have not yet been processed and a plurality of semi-finished molds that have been processed,” “the tool placement module including a tool carrying frame and a second movable safety door adjacent to the tool carrying frame, the tool carrying frame being configured to carry a plurality of processing tools, and each of the processing tools including a tool holder and a tool detachably clamped by the tool holder,” “the material processing module including a plurality of material processing machines, and each of the material processing machines being configured to use one of the processing tools to process one of the mold materials, thereby obtaining a corresponding one of the semi-finished molds,” and “the mold cleaning module including a first cleaning tank for accommodating liquid medicine, a second cleaning tank for accommodating clear water and a drying tank for providing hot air,” the mold manufacturing system provided by the present disclosure can realize the production of molds in an automated manner.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a mold manufacturing system according to a first embodiment of the present disclosure;

FIG. 2 is a schematic view of a robot arm module and a mold placement module of the mold manufacturing system cooperated with each other according to the first embodiment of the present disclosure;

FIG. 3 is a schematic view of a mold carrying frame configured to carry a plurality of mold materials that have not yet been processed and a plurality of semi-finished molds that have been processed in the mold manufacturing system according to the first embodiment of the present disclosure;

FIG. 4 is a schematic view of the robot arm module and a tool placement module of the mold manufacturing system cooperated with each other according to the first embodiment of the present disclosure;

FIG. 5 is a schematic view of a tool carrying frame configured to carry a plurality of processing tools in the mold manufacturing system according to the first embodiment of the present disclosure;

FIG. 6 is a schematic view of the tool placement module of the mold manufacturing system including a rotatable carrier for carrying a tool holder clamping a tool and a barcode reader adjacent to the rotatable carrier according to the first embodiment of the present disclosure;

FIG. 7 is a schematic view of the barcode reader configured for reading the tool identification barcode of the tool holder of the tool placement module of the mold manufacturing system according to the first embodiment of the present disclosure;

FIG. 8 is a schematic view of the robot arm module and a mold cleaning module of the mold manufacturing system cooperated with each other according to the first embodiment of the present disclosure;

FIG. 9 is a functional block diagram of the mold manufacturing system according to a second embodiment of the present disclosure; and

FIG. 10 is a functional block diagram of the mold manufacturing system of a third embodiment of the present invention.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

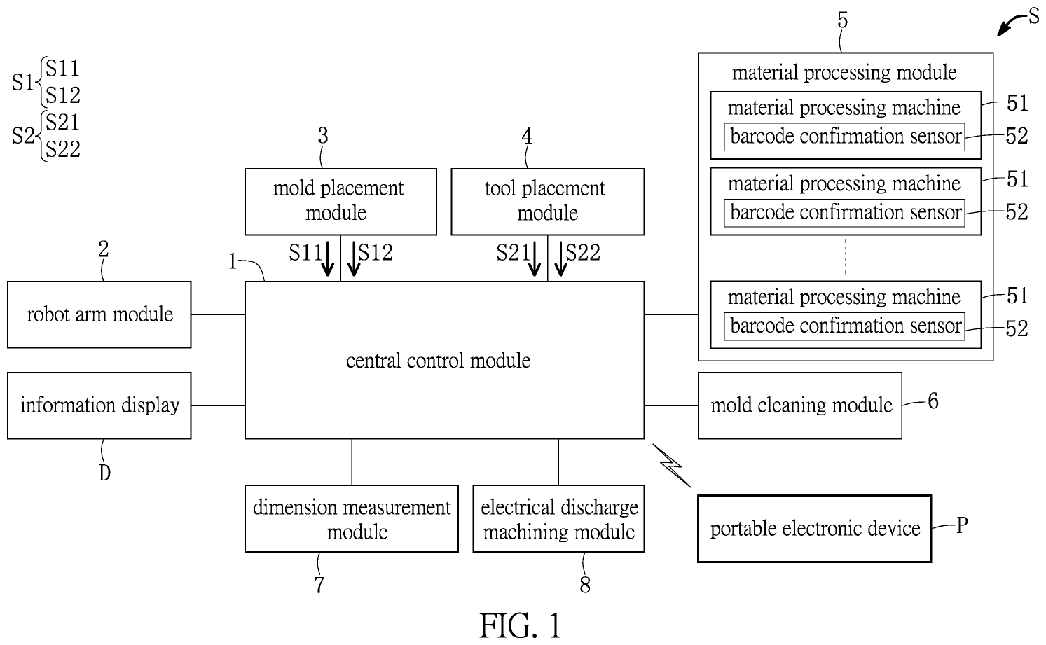

Referring to FIG. 1 to FIG. 8, a first embodiment of the present disclosure provides a mold manufacturing system S, which includes a central control module 1, a robot arm module 2, a mold placement module 3 (or a mold storage module), a tool placement module 4 (or a tool storage module), a material processing module 5, a mold cleaning module 6, a dimension measurement module 7 and an electrical discharge machining module 8, and the robot arm module 2, the mold placement module 3, the tool placement module 4, the material processing module 5, the mold cleaning module 6, the dimension measurement module 7 and the electrical discharge machining module 8 are all electrically connected to the central control module 1, so that the central control module 1 can be configured to control the robot arm module 2, the mold placement module 3, the tool placement module 4, the material processing module 5, the mold cleaning module 6, the dimension measurement module 7 and the electrical discharge machining module 8.

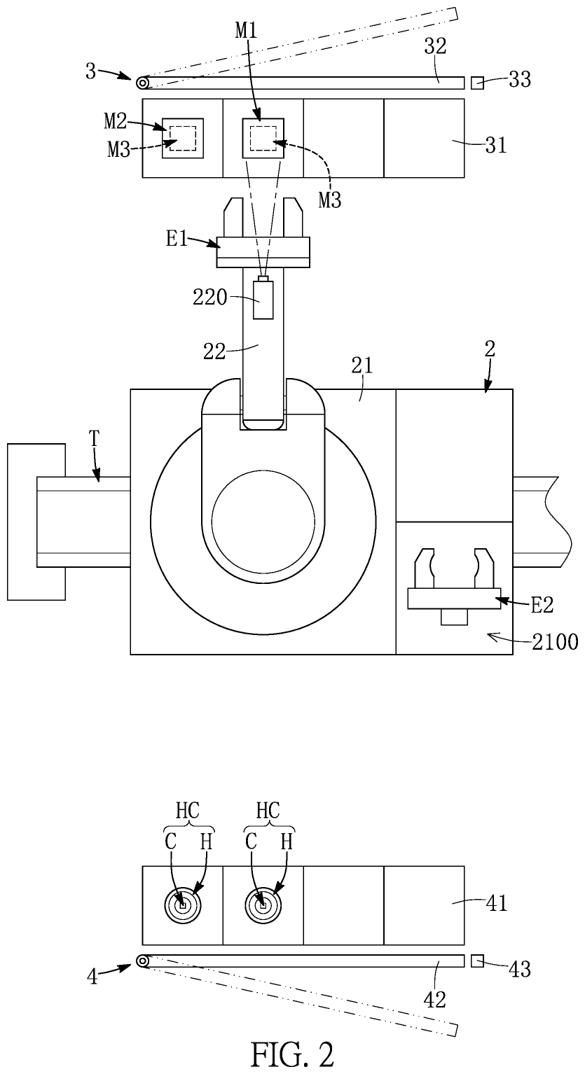

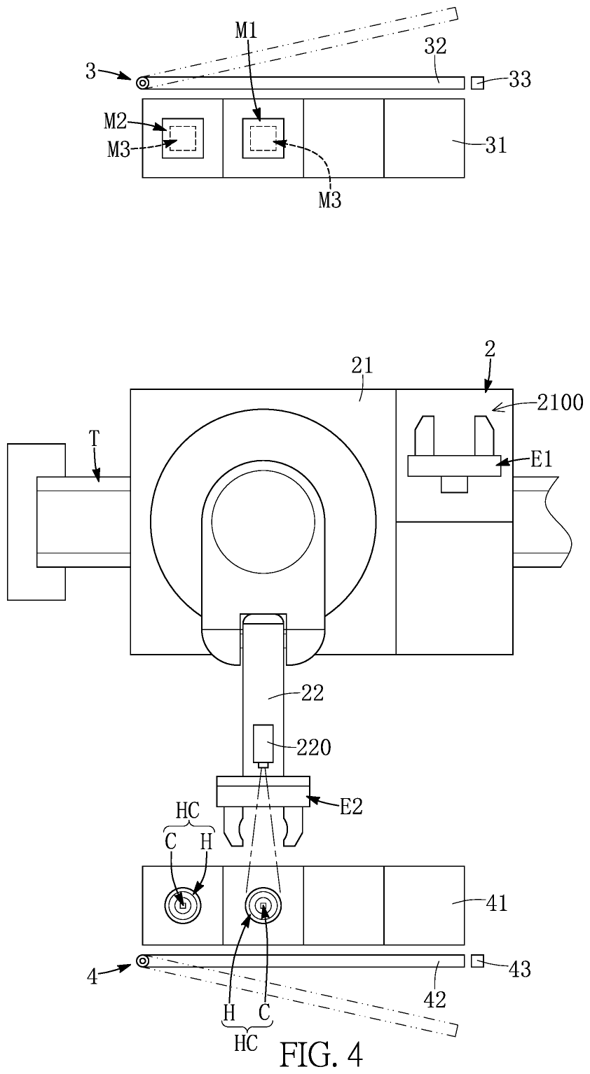

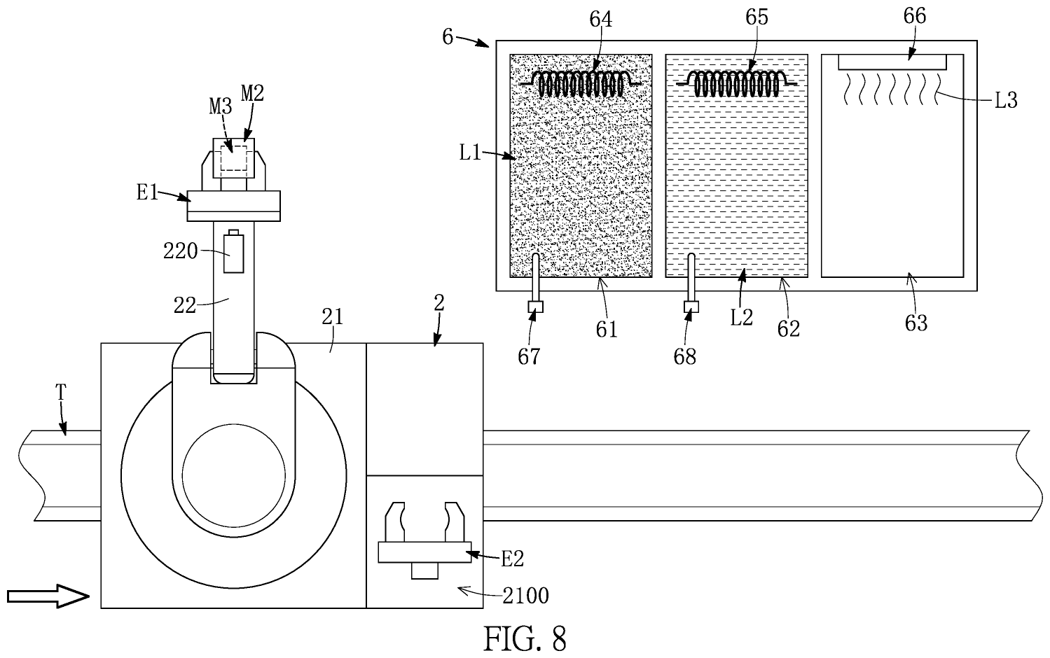

Firstly, referring to FIG. 1, FIG. 2, FIG. 4 and FIG. 8, the robot arm module 2 is movably disposed on a system track T. For example, the mold placement module 3, the tool placement module 4, the material processing module 5, the mold cleaning module 6, the dimension measurement module 7 and the electrical discharge machining module 8 are all adjacent to the system track T and arranged along the system track T, so that the robot arm module 2 can be configured to be movably disposed among the mold placement module 3, the tool placement module 4, the material processing module 5, the mold cleaning module 6, the dimension measurement module 7 and the electrical discharge machining module 8. That is to say, the robot arm module 2 can move along the system track T to facilitate delivery of items among the mold placement module 3, the tool placement module 4, the material processing module 5, the mold cleaning module 6, the dimension measurement module 7 and the electrical discharge machining module 8. However, the aforementioned details are disclosed for exemplary purposes only, and are not meant to limit the scope of the present disclosure.

More particularly, referring to FIG. 1, FIG. 2, FIG. 4 and FIG. 8, the robot arm module 2 includes a movable base 21 movably disposed on the system track T and a robot arm 22 movably disposed on the movable base 21. It should be noted that, the movable base 21 has a gripper accommodating space 2100 for accommodating at least one mold gripping jaw E1 and at least one tool gripping jaw E2, and the robot arm 22 has a barcode reader 220. For example, as shown in FIG. 2, when the mold gripping jaw E1 is in use, the tool gripping jaw E2 is accommodated in the gripper accommodating space 2100. As shown in FIG. 4, when the tool gripping jaw E2 is in use, the mold gripping jaw E1 is accommodated in the gripper accommodating space 2100. In other words, as shown in FIG. 2 or FIG. 4, when one of the at least one mold gripping jaw E1 and the at least one tool gripping jaw E2 is detachably connected to the robot arm 22, another one of the at least one mold gripping jaw E1 and the at least one tool gripping jaw E2 can be accommodated in the gripper accommodating space 2100 of the movable base 21. However, the aforementioned details are disclosed for exemplary purposes only, and are not meant to limit the scope of the present disclosure.

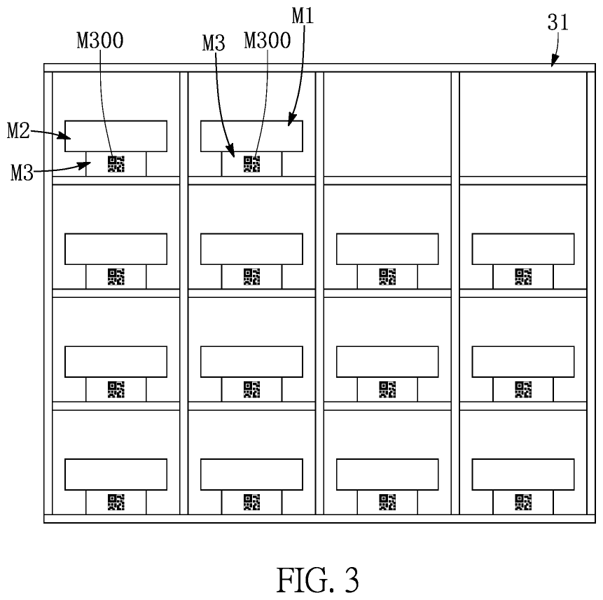

Furthermore, referring to FIG. 1, FIG. 2 and FIG. 3, the mold placement module 3 includes a mold carrying frame 31 and a first movable safety door 32 adjacent to the mold carrying frame 31, and the mold carrying frame 31 can be configured to carry a plurality of mold materials M1 (i.e., a plurality of unprocessed mold materials M1) that have not yet been processed and a plurality of semi-finished molds M2 (i.e., a plurality of processed semi-finished molds M2) that have been processed. More particularly, the mold placement module 3 includes a first sensor 33 adjacent to the first movable safety door 32, and the first sensor 33 can be configured to determine or judge whether the first movable safety door 32 is in an open state or a closed state. In addition, the mold materials M1 and the semi-finished molds M2 are respectively and detachably disposed on a plurality of corresponding mold carrying seats M3 (or mold carriers), and each of the mold carrying seats M3 has a mold identification barcode M300 that can be configured to be read by the barcode reader 220 of the robot arm 22, and mold information provided by the mold identification barcode M300 of the mold carrying seat M3 can correspond to a type of the mold material M1 or a type of the semi-finished mold M2 that is carried by the mold carrying seat M3.

For example, as shown in FIG. 1, the mold placement module 3 can be configured to provide mold material information S1 and semi-finished mold information S2 to the central control module 1, the mold material information S1 includes mold material quantity information S11 and mold material location information S12, and the semi-finished mold information S2 includes semi-finished mold quantity information S21 and semi-finished mold location information S22. In addition, the mold material quantity information S11 and the mold material location information S12 of the mold material information S1 and the semi-finished mold quantity information S21 and the semi-finished mold location information S22 of the semi-finished mold information S2 are all displayed on an information display D that is electrically connected to the central control module 1, and the mold material quantity information S11 and the mold material location information S12 of the mold material information S1 and the semi-finished mold quantity information S21 and the semi-finished mold location information S22 of the semi-finished mold information S2 can be transmitted to a portable electronic device P carried by a user through the central control module 1 (such as by wireless communication). However, the aforementioned details are disclosed for exemplary purposes only, and are not meant to limit the scope of the present disclosure.

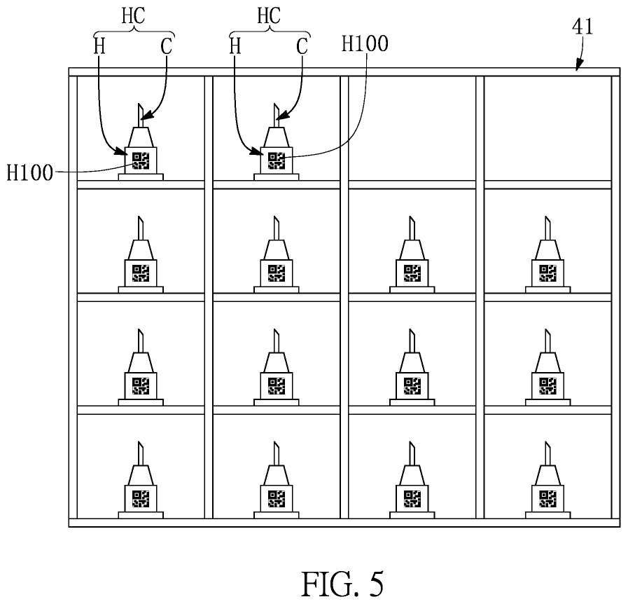

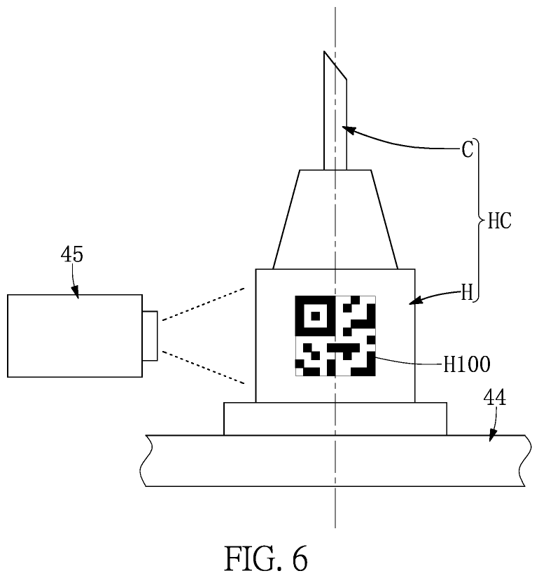

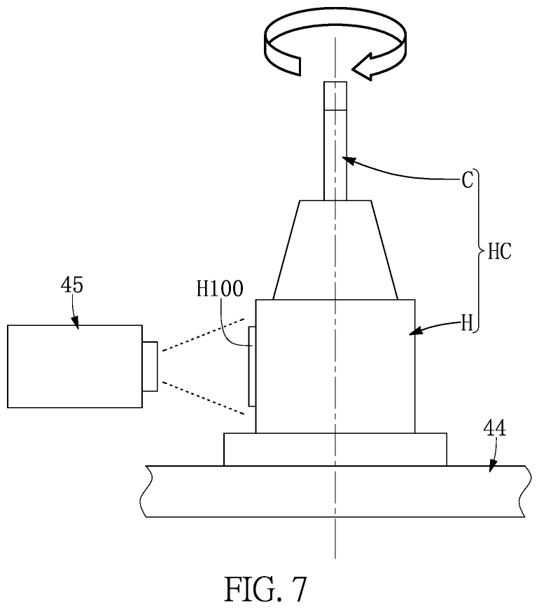

Moreover, referring to FIG. 1, FIG. 4 and FIG. 5, the tool placement module 4 includes a tool carrying frame 41 and a second movable safety door 42 adjacent to the tool carrying frame 41, the tool carrying frame 41 can be configured to carry a plurality of processing tools HC, and each of the processing tools HC includes a tool holder H (or a tool handle) and a tool C detachably clamped or held by the tool holder H. More particularly, the tool placement module 4 includes a second sensor 43 adjacent to the second movable safety door 42, and the second sensor 43 can be configured to determine or judge whether the second movable safety door 42 is in an opened state or a closed state. In addition, the tool holder H has a tool identification barcode H100 that can be configured to be read by the barcode reader 220 of the robot arm 22, and tool information provided by the tool identification barcode H100 of the tool holder H can correspond to a type of the tool C that is clamped or holed by the tool holder H. It should be noted that referring to FIG. 6 and FIG. 7, the tool placement module 4 further includes a rotatable carrier 44 (or a rotatable platform) for carrying the tool holder H clamping or holding the tool C (i.e., the processing tool HC including the tool holder H and the tool C), and a tool holder barcode reader 45 adjacent to the rotatable carrier 44. For example, when the tool holder H clamping the tool C is rotated by rotating of the rotatable carrier 44 so as to rotate the tool identification barcode H100 of the tool holder H to face the tool holder barcode reader 45 (as shown in FIG. 7), the tool holder barcode reader 45 can be configured to read the tool identification barcode H100 of the tool holder H. However, the aforementioned details are disclosed for exemplary purposes only, and are not meant to limit the scope of the present disclosure.

In addition, as shown in FIG. 1, the material processing module 5 includes a plurality of material processing machines 51, and each of the material processing machines 51 can be configured to use one of the processing tools HC to process one of the mold materials M1, thereby obtaining a corresponding one of the semi-finished molds M2. More particularly, the material processing module 5 includes a plurality of barcode confirmation sensors 52 respectively disposed inside the material processing machines 51, and each of the barcode confirmation sensors 52 can be configured to read the mold identification barcode M300 of the mold carrying seat M3 and the tool identification barcode H100 of the tool holder H that are disposed inside the corresponding material processing machine 51, thereby confirming whether or not the mold material M1 and the tool C used in the material processing machine 51 is correct.

Furthermore, referring to FIG. 1 and FIG. 8, the mold cleaning module 6 includes a first cleaning tank 61 for accommodating liquid medicine L1, a second cleaning tank 62 for accommodating clear water L2 and a drying tank 63 for providing hot air L3. More particularly, the mold cleaning module 6 further includes a first heater 64 configured to provide heat source for the first cleaning tank 61 (i.e., using the first heater 64 to heat the liquid medicine L1), a second heater 65 configured to provide heat source for the second cleaning tank 62 (i.e., using the second heater 65 to heat the clear water L2), and a hot air provider 66 configured to provide heat source for the drying tank 63 (i.e., using the hot air provider 66 to provide hot air L3), and the first heater 64 and the second heater 65 can also be connected and combined into the same heater. Moreover, the mold cleaning module 6 further includes a first water quality detector 67 disposed in the first cleaning tank 61 and a second water quality detector 68 disposed in the second cleaning tank 62. The first water quality detector 67 can be configured to detect a water quality of the liquid medicine L1 in the first cleaning tank 61 (for example, first water quality information can be provided to the central control module 1 to determine whether it is necessary to replace the liquid medicine L1 in the first cleaning tank 61, and record the time when the liquid medicine L1 is used to clean the semi-finished mold M2, and when it is necessary to replace the liquid medicine L1 in the first cleaning tank 61, automatically discharging the liquid medicine L1 from the first cleaning tank 61 and then automatically injecting new liquid medicine into the first cleaning tank 61), and the second water quality detector 68 can be configured to detect a water quality of the clear water L2 in the second cleaning tank 62 (for example, second water quality information can be provided to the central control module 1 to determine whether it is necessary to replace the clean water L2 in the second cleaning tank 62, and record the time when the clean water L2 is used to clean the semi-finished mold M2, and when it is necessary to replace the clean water L2 in the second cleaning tank 62, automatically discharging the clean water L2 from the second cleaning tank 62 and then automatically injecting new clean water into the second cleaning tank 62). For example, when the robot arm 22 of the robot arm module 2 is configured to use the at least one mold gripping jaw E1, the mold carrying seat M3 carrying the semi-finished mold M2 can be immersed into the first cleaning tank 61 containing the liquid medicine L1 by clamping of the at least one mold gripping jaw E1, so that the semi-finished mold M2 can be cleaned by the liquid medicine L1 through ultrasonic vibration (e.g., using an ultrasonic generator). When the robot arm 22 of the robot arm module 2 is configured to use the at least one mold gripping jaw E1, the mold carrying seat M3 carrying the semi-finished mold M2 can be transferred from the first cleaning tank 61 to the second cleaning tank 62 and immersed into the second cleaning tank 62 containing the clear water L2 by clamping of the at least one mold gripping jaw E1, so that the semi-finished mold M2 can be cleaned by the clear water L2 through ultrasonic vibration (e.g., using an ultrasonic generator). When the robot arm 22 of the robot arm module 2 is configured to use the at least one mold gripping jaw E1, the mold carrying seat M3 carrying the semi-finished mold M2 can be transferred from the second cleaning tank 62 to the drying tank 63 by clamping of the at least one mold gripping jaw E1, so that the semi-finished mold M2 can be dried by the hot air L3 provided by the drying tank 63 so as to remove excess moisture. It should be noted that the mold cleaning module 6 also includes a barcode reader (not shown in the figure), which is used to confirm the relevant information of the semi-finished mold M2 by reading the mold identification barcode M300 of the mold carrying seat M3. However, the aforementioned details are disclosed for exemplary purposes only, and are not meant to limit the scope of the present disclosure.

In addition, as shown in FIG. 1, the dimension measurement module 7 can be configured to measure the semi-finished mold M2, thereby obtaining actual dimension information (such as the length, width and height of different structures) of the semi-finished mold M2. For example, the dimension measurement module 7 includes an image dimension measuring instrument, a surface 3D profile measuring instrument or a coordinate measuring machine. However, the aforementioned details are disclosed for exemplary purposes only, and are not meant to limit the scope of the present disclosure.

Moreover, as shown in FIG. 1, the electrical discharge machining module 8 can be configured to perform precision machining on the semi-finished mold M2 according to a difference between the actual dimension information (i.e., processing size information) and standard dimension information (i.e., target size information), thereby making the difference between the actual dimension information of the semi-finished mold M2 and the standard dimension information is within a predetermined error range (such as the error range between 10 and 0.1 μm, or 0.1 and 0.01 μm). For example, after the actual dimension information of the semi-finished mold M2 is transmitted from the dimension measurement module 7 to the central control module 1, the electrical discharge machining module 8 can be controlled by the central control module 1 for precisely processing the semi-finished mold M2 according to the actual dimension information. However, the aforementioned details are disclosed for exemplary purposes only, and are not meant to limit the scope of the present disclosure.

### Second Embodiment

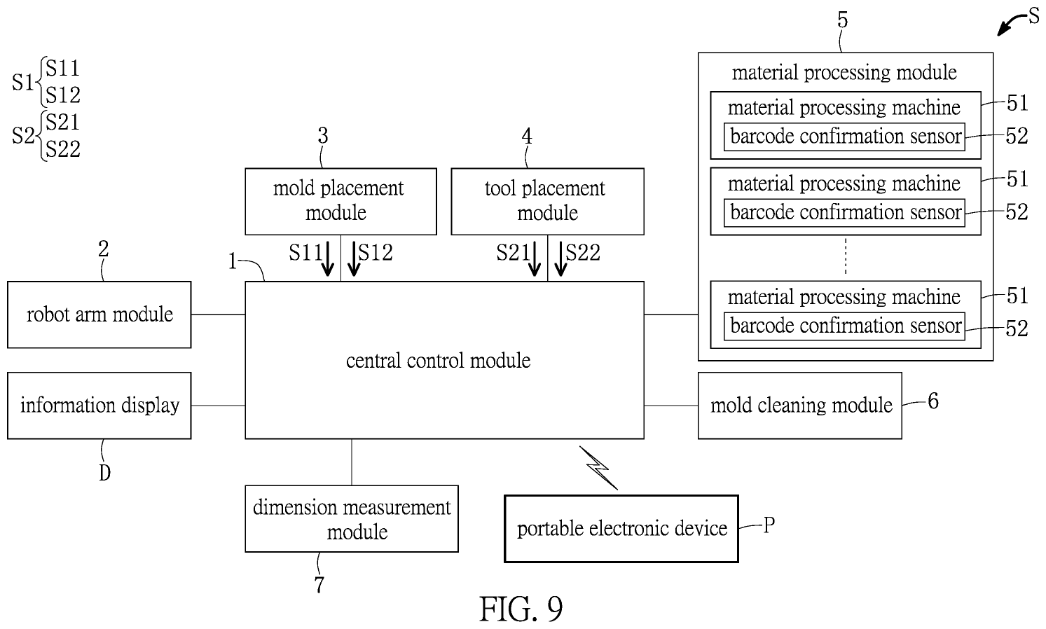

Referring to FIG. 1 to FIG. 9, a second embodiment of the present disclosure provides a mold manufacturing system S, which includes a central control module 1, a robot arm module 2, a mold placement module 3, a tool placement module 4, a material processing module 5, a mold cleaning module 6 and a dimension measurement module 7. Comparing FIG. 9 with FIG. 1, the main difference between the second embodiment and the first embodiment is as follows: in the second embodiment, the electrical discharge machining module 8 used in the first embodiment can be incorporated in the mold manufacturing system S in a non-fully automated manner. In other words, the semi-finished mold M2 can be manually transmitted to the electrical discharge machining module 8 for processing by a user without using the robot arm module 2.

### Third Embodiment

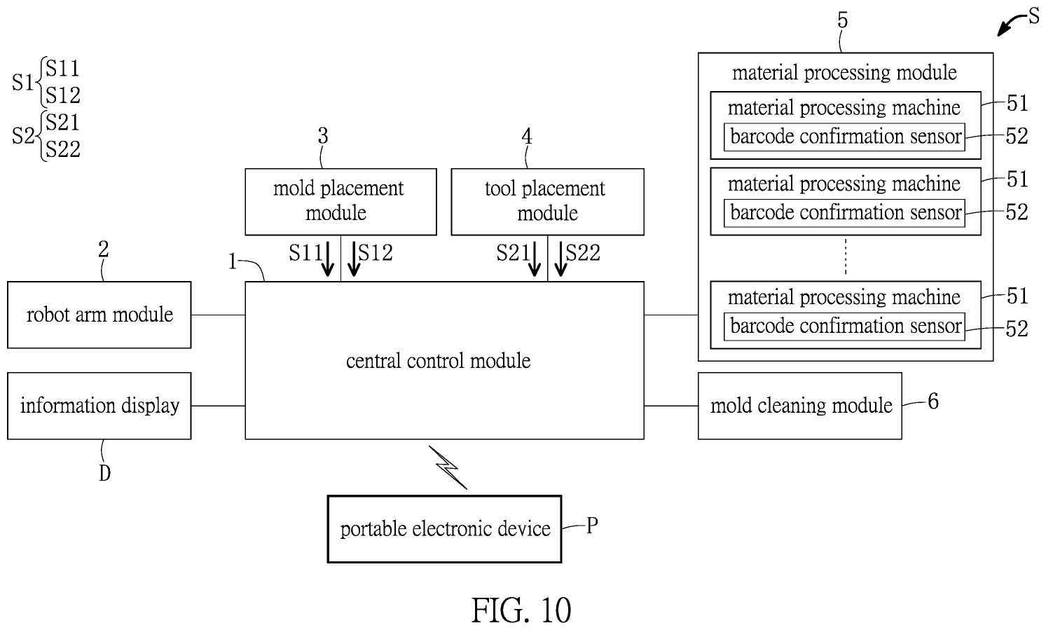

Referring to FIG. 1 to FIG. 10, a third embodiment of the present disclosure provides a mold manufacturing system S, which includes a central control module 1, a robot arm module 2, a mold placement module 3, a tool placement module 4, a material processing module 5 and a mold cleaning module 6. Comparing FIG. 10 with FIG. 1, the main difference between the third embodiment and the first embodiment is as follows: in the third embodiment, the dimension measurement module 7 and the electrical discharge machining module 8 used in the first embodiment can be incorporated in the mold manufacturing system S in a non-fully automated manner. In other words, the semi-finished mold M2 can be manually transmitted to the dimension measurement module 7 and the electrical discharge machining module 8 for measuring and processing by a user without using the robot arm module 2.

### Beneficial Effects of the Embodiments

In conclusion, in the mold manufacturing system S provided by the present disclosure, by virtue of “the robot arm module 2 being movably disposed on a system track T,” “the mold placement module 3 including a mold carrying frame 31 and a first movable safety door 32 adjacent to the mold carrying frame 31, and the mold carrying frame 31 being configured to carry a plurality of mold materials M1 that have not yet been processed and a plurality of semi-finished molds M2 that have been processed,” “the tool placement module 4 including a tool carrying frame 41 and a second movable safety door 42 adjacent to the tool carrying frame 41, the tool carrying frame 41 being configured to carry a plurality of processing tools HC, and each of the processing tools HC including a tool holder H and a tool detachably clamped by the tool holder H,” “the material processing module 5 including a plurality of material processing machines 51, and each of the material processing machines 51 being configured to use one of the processing tools HC to process one of the mold materials M1, thereby obtaining a corresponding one of the semi-finished molds M2,” and “the mold cleaning module 6 including a first cleaning tank 61 for accommodating liquid medicine L1, a second cleaning tank 62 for accommodating clear water L2 and a drying tank 63 for providing hot air L3,” the mold manufacturing system S provided by the present disclosure can realize the production of molds in an automated manner.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A mold manufacturing system, comprising:
a central control module;
a robot arm module electrically connected to the central control module, wherein the robot arm module is movably disposed on a system track;
a mold placement module electrically connected to the central control module, wherein the mold placement module includes a mold carrying frame and a first movable safety door adjacent to the mold carrying frame, and the mold carrying frame is configured to carry a plurality of mold materials that have not yet been processed and a plurality of semi-finished molds that have been processed;
a tool placement module electrically connected to the central control module, wherein the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder;
a material processing module electrically connected to the central control module, wherein the material processing module includes a plurality of material processing machines, and each of the material processing machines is configured to use one of the processing tools to process one of the mold materials, thereby obtaining a corresponding one of the semi-finished molds;
a mold cleaning module electrically connected to the central control module, wherein the mold cleaning module includes a first cleaning tank for accommodating liquid medicine, a second cleaning tank for accommodating clear water and a drying tank for providing hot air;
a dimension measurement module electrically connected to the central control module, wherein the dimension measurement module is configured to measure the semi-finished mold, thereby obtaining actual dimension information of the semi-finished mold; and
an electrical discharge machining module electrically connected to the central control module, wherein the electrical discharge machining module is configured to perform precision machining on the semi-finished mold according to a difference between the actual dimension information and standard dimension information, thereby making the difference between the actual dimension information of the semi-finished mold and the standard dimension information is within a predetermined error range;
wherein the mold placement module, the tool placement module, the material processing module, the mold cleaning module, the dimension measurement module and the electrical discharge machining module are all adjacent to the system track and arranged along the system track;
wherein the robot arm module is configured to be movably disposed among the mold placement module, the tool placement module, the material processing module, the mold cleaning module, the dimension measurement module and the electrical discharge machining module and along the system track.

2. The mold manufacturing system according to claim 1,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein the mold placement module is configured to provide mold material information and semi-finished mold information to the central control module, the mold material information includes mold material quantity information and mold material location information, and the semi-finished mold information includes semi-finished mold quantity information and semi-finished mold location information;
wherein the mold material quantity information and the mold material location information of the mold material information and the semi-finished mold quantity information and the semi-finished mold location information of the semi-finished mold information are all displayed on an information display electrically connected to the central control module, and the mold material quantity information and the mold material location information of the mold material information and the semi-finished mold quantity information and the semi-finished mold location information of the semi-finished mold information are transmitted to a portable electronic device of a user through the central control module;
wherein the mold placement module includes a first sensor adjacent to the first movable safety door, and the first sensor is configured to determine whether the first movable safety door is in an open state or a closed state;
wherein the tool placement module includes a second sensor adjacent to the second movable safety door, and the second sensor is configured to determine whether the second movable safety door is in an opened state or a closed state.

3. The mold manufacturing system according to claim 1,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein, the mold materials and the semi-finished molds are respectively and detachably disposed on a plurality of corresponding mold carrying seats, and each of the mold carrying seats has a mold identification barcode that is configured to be read by the barcode reader of the robot arm, and mold information provided by the mold identification barcode of the mold carrying seat corresponds to a type of the mold material or a type of the semi-finished mold that is carried by the mold carrying seat;
wherein the tool holder has a tool identification barcode that is configured to be read by the barcode reader of the robot arm, and tool information provided by the tool identification barcode of the tool holder corresponds to a type of the tool clamped by the tool holder;
wherein the tool placement module further includes a rotatable carrier for carrying the tool holder clamping the tool, and a tool holder barcode reader adjacent to the rotatable carrier;
wherein, when the tool holder clamping the tool is rotated by rotating of the rotatable carrier so as to rotate the tool identification barcode of the tool holder to face the tool holder barcode reader, the tool holder barcode reader is configured to read the tool identification barcode of the tool holder;
wherein, when the robot arm of the robot arm module is configured to use the at least one mold gripping jaw, the mold carrying seat carrying the semi-finished mold is immersed into the first cleaning tank containing the liquid medicine by clamping of the at least one mold gripping jaw, so that the semi-finished mold is cleaned by the liquid medicine through ultrasonic vibration;
wherein, when the robot arm of the robot arm module is configured to use the at least one mold gripping jaw, the mold carrying seat carrying the semi-finished mold is transferred from the first cleaning tank to the second cleaning tank and immersed into the second cleaning tank containing the clear water by clamping of the at least one mold gripping jaw, so that the semi-finished mold is cleaned by the clear water through ultrasonic vibration;
wherein, when the robot arm of the robot arm module is configured to use the at least one mold gripping jaw, the mold carrying seat carrying the semi-finished mold is transferred from the second cleaning tank to the drying tank by clamping of the at least one mold gripping jaw, so that the semi-finished mold is dried by the hot air provided by the drying tank so as to remove excess moisture;
wherein the mold cleaning module further includes a first heater configured to provide heat source for the first cleaning tank, a second heater configured to provide heat source for the second cleaning tank, and a hot air provider configured to provide heat source for the drying tank;
wherein the material processing module includes a plurality of barcode confirmation sensors respectively disposed inside the material processing machines, and each of the barcode confirmation sensors is configured to read the mold identification barcode of the mold carrying seat and the tool identification barcode of the tool holder in the material processing machine, thereby confirming whether or not the mold material and the tool used in the material processing machine is correct;
wherein the mold cleaning module further includes a first water quality detector disposed in the first cleaning tank and a second water quality detector disposed in the second cleaning tank, the first water quality detector is configured to detect a water quality of the liquid medicine in the first cleaning tank, and the second water quality detector is configured to detect a water quality of the clear water in the second cleaning tank;
wherein the dimension measurement module includes an image dimension measuring instrument, a surface 3D profile measuring instrument or a coordinate measuring machine;
wherein, after the actual dimension information of the semi-finished mold is transmitted from the dimension measurement module to the central control module, the electrical discharge machining module is controlled by the central control module for precisely processing the semi-finished mold according to the actual dimension information.

4. A mold manufacturing system, comprising:
a central control module;
a robot arm module electrically connected to the central control module, wherein the robot arm module is movably disposed on a system track;
a mold placement module electrically connected to the central control module, wherein the mold placement module includes a mold carrying frame and a first movable safety door adjacent to the mold carrying frame, and the mold carrying frame is configured to carry a plurality of mold materials that have not yet been processed and a plurality of semi-finished molds that have been processed;
a tool placement module electrically connected to the central control module, wherein the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder;
a material processing module electrically connected to the central control module, wherein the material processing module includes a plurality of material processing machines, and each of the material processing machines is configured to use one of the processing tools to process one of the mold materials, thereby obtaining a corresponding one of the semi-finished molds;
a mold cleaning module electrically connected to the central control module, wherein the mold cleaning module includes a first cleaning tank for accommodating liquid medicine, a second cleaning tank for accommodating clear water and a drying tank for providing hot air; and
a dimension measurement module electrically connected to the central control module, wherein the dimension measurement module is configured to measure the semi-finished mold, thereby obtaining actual dimension information of the semi-finished mold;
wherein the mold placement module, the tool placement module, the material processing module, the mold cleaning module and the dimension measurement module are all adjacent to the system track and arranged along the system track;
wherein the robot arm module is configured to be movably disposed among the mold placement module, the tool placement module, the material processing module, the mold cleaning module and the dimension measurement module and along the system track.

5. The mold manufacturing system according to claim 4,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein the mold placement module is configured to provide mold material information and semi-finished mold information to the central control module, the mold material information includes mold material quantity information and mold material location information, and the semi-finished mold information includes semi-finished mold quantity information and semi-finished mold location information;
wherein the mold material quantity information and the mold material location information of the mold material information and the semi-finished mold quantity information and the semi-finished mold location information of the semi-finished mold information are all displayed on an information display electrically connected to the central control module, and the mold material quantity information and the mold material location information of the mold material information and the semi-finished mold quantity information and the semi-finished mold location information of the semi-finished mold information are transmitted to a portable electronic device of a user through the central control module;
wherein the mold placement module includes a first sensor adjacent to the first movable safety door, and the first sensor is configured to determine whether the first movable safety door is in an open state or a closed state;
wherein the tool placement module includes a second sensor adjacent to the second movable safety door, and the second sensor is configured to determine whether the second movable safety door is in an opened state or a closed state.

6. The mold manufacturing system according to claim 4,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein, the mold materials and the semi-finished molds are respectively and detachably disposed on a plurality of corresponding mold carrying seats, and each of the mold carrying seats has a mold identification barcode that is configured to be read by the barcode reader of the robot arm, and mold information provided by the mold identification barcode of the mold carrying seat corresponds to a type of the mold material or a type of the semi-finished mold that is carried by the mold carrying seat;
wherein the tool holder has a tool identification barcode that is configured to be read by the barcode reader of the robot arm, and tool information provided by the tool identification barcode of the tool holder corresponds to a type of the tool clamped by the tool holder;
wherein the tool placement module further includes a rotatable carrier for carrying the tool holder clamping the tool, and a tool holder barcode reader adjacent to the rotatable carrier;
wherein, when the tool holder clamping the tool is rotated by rotating of the rotatable carrier so as to rotate the tool identification barcode of the tool holder to face the tool holder barcode reader, the tool holder barcode reader is configured to read the tool identification barcode of the tool holder;
wherein, when the robot arm of the robot arm module is configured to use the at least one mold gripping jaw, the mold carrying seat carrying the semi-finished mold is immersed into the first cleaning tank containing the liquid medicine by clamping of the at least one mold gripping jaw, so that the semi-finished mold is cleaned by the liquid medicine through ultrasonic vibration;
wherein, when the robot arm of the robot arm module is configured to use the at least one mold gripping jaw, the mold carrying seat carrying the semi-finished mold is transferred from the first cleaning tank to the second cleaning tank and immersed into the second cleaning tank containing the clear water by clamping of the at least one mold gripping jaw, so that the semi-finished mold is cleaned by the clear water through ultrasonic vibration;
wherein, when the robot arm of the robot arm module is configured to use the at least one mold gripping jaw, the mold carrying seat carrying the semi-finished mold is transferred from the second cleaning tank to the drying tank by clamping of the at least one mold gripping jaw, so that the semi-finished mold is dried by the hot air provided by the drying tank so as to remove excess moisture;
wherein the mold cleaning module further includes a first heater configured to provide heat source for the first cleaning tank, a second heater configured to provide heat source for the second cleaning tank, and a hot air provider configured to provide heat source for the drying tank;
wherein the material processing module includes a plurality of barcode confirmation sensors respectively disposed inside the material processing machines, and each of the barcode confirmation sensors is configured to read the mold identification barcode of the mold carrying seat and the tool identification barcode of the tool holder in the material processing machine, thereby confirming whether or not the mold material and the tool used in the material processing machine is correct;
wherein the mold cleaning module further includes a first water quality detector disposed in the first cleaning tank and a second water quality detector disposed in the second cleaning tank, the first water quality detector is configured to detect a water quality of the liquid medicine in the first cleaning tank, and the second water quality detector is configured to detect a water quality of the clear water in the second cleaning tank;
wherein the dimension measurement module includes an image dimension measuring instrument, a surface 3D profile measuring instrument or a coordinate measuring machine.

7. A mold manufacturing system, comprising:
a central control module;
a robot arm module electrically connected to the central control module, wherein the robot arm module is movably disposed on a system track;
a mold placement module electrically connected to the central control module, wherein the mold placement module includes a mold carrying frame and a first movable safety door adjacent to the mold carrying frame, and the mold carrying frame is configured to carry a plurality of mold materials that have not yet been processed and a plurality of semi-finished molds that have been processed;
a tool placement module electrically connected to the central control module, wherein the tool placement module includes a tool carrying frame and a second movable safety door adjacent to the tool carrying frame, the tool carrying frame is configured to carry a plurality of processing tools, and each of the processing tools includes a tool holder and a tool detachably clamped by the tool holder;
a material processing module electrically connected to the central control module, wherein the material processing module includes a plurality of material processing machines, and each of the material processing machines is configured to use one of the processing tools to process one of the mold materials, thereby obtaining a corresponding one of the semi-finished molds; and
a mold cleaning module electrically connected to the central control module, wherein the mold cleaning module includes a first cleaning tank for accommodating liquid medicine, a second cleaning tank for accommodating clear water and a drying tank for providing hot air;
wherein the mold placement module, the tool placement module, the material processing module and the mold cleaning module are all adjacent to the system track and arranged along the system track;
wherein the robot arm module is configured to be movably disposed among the mold placement module, the tool placement module, the material processing module and the mold cleaning module and along the system track.

8. The mold manufacturing system according to claim 7,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein the mold placement module is configured to provide mold material information and semi-finished mold information to the central control module, the mold material information includes mold material quantity information and mold material location information, and the semi-finished mold information includes semi-finished mold quantity information and semi-finished mold location information;
wherein the mold material quantity information and the mold material location information of the mold material information and the semi-finished mold quantity information and the semi-finished mold location information of the semi-finished mold information are all displayed on an information display electrically connected to the central control module, and the mold material quantity information and the mold material location information of the mold material information and the semi-finished mold quantity information and the semi-finished mold location information of the semi-finished mold information are transmitted to a portable electronic device of a user through the central control module;
wherein the mold placement module includes a first sensor adjacent to the first movable safety door, and the first sensor is configured to determine whether the first movable safety door is in an open state or a closed state;
wherein the tool placement module includes a second sensor adjacent to the second movable safety door, and the second sensor is configured to determine whether the second movable safety door is in an opened state or a closed state.

9. The mold manufacturing system according to claim 7,
wherein the robot arm module includes a movable base movably disposed on the system track and a robot arm movably disposed on the movable base, the movable base has a gripper accommodating space for accommodating at least one mold gripping jaw and at least one tool gripping jaw, and the robot arm has a barcode reader;
wherein, when one of the at least one mold gripping jaw and the at least one tool gripping jaw is detachably connected to the robot arm, another one of the at least one mold gripping jaw and the at least one tool gripping jaw is accommodated in the gripper accommodating space of the movable base;
wherein, the mold materials and the semi-finished molds are respectively and detachably disposed on a plurality of corresponding mold carrying seats, and each of the mold carrying seats has a mold identification barcode that is configured to be read by the barcode reader of the robot arm, and mold information provided by the mold identification barcode of the mold carrying seat corresponds to a type of the mold material or a type of the semi-finished mold that is carried by the mold carrying seat;
wherein the tool holder has a tool identification barcode that is configured to be read by the barcode reader of the robot arm, and tool information provided by the tool identification barcode of the tool holder corresponds to a type of the tool clamped by the tool holder;
wherein the tool placement module further includes a rotatable carrier for carrying the tool holder clamping the tool, and a tool holder barcode reader adjacent to the rotatable carrier;
wherein, when the tool holder clamping the tool is rotated by rotating of the rotatable carrier so as to rotate the tool identification barcode of the tool holder to face the tool holder barcode reader, the tool holder barcode reader is configured to read the tool identification barcode of the tool holder;
wherein, when the robot arm of the robot arm module is configured to use the at least one mold gripping jaw, the mold carrying seat carrying the semi-finished mold is immersed into the first cleaning tank containing the liquid medicine by clamping of the at least one mold gripping jaw, so that the semi-finished mold is cleaned by the liquid medicine through ultrasonic vibration;
wherein, when the robot arm of the robot arm module is configured to use the at least one mold gripping jaw, the mold carrying seat carrying the semi-finished mold is transferred from the first cleaning tank to the second cleaning tank and immersed into the second cleaning tank containing the clear water by clamping of the at least one mold gripping jaw, so that the semi-finished mold is cleaned by the clear water through ultrasonic vibration;
wherein, when the robot arm of the robot arm module is configured to use the at least one mold gripping jaw, the mold carrying seat carrying the semi-finished mold is transferred from the second cleaning tank to the drying tank by clamping of the at least one mold gripping jaw, so that the semi-finished mold is dried by the hot air provided by the drying tank so as to remove excess moisture;
wherein the mold cleaning module further includes a first heater configured to provide heat source for the first cleaning tank, a second heater configured to provide heat source for the second cleaning tank, and a hot air provider configured to provide heat source for the drying tank;
wherein the material processing module includes a plurality of barcode confirmation sensors respectively disposed inside the material processing machines, and each of the barcode confirmation sensors is configured to read the mold identification barcode of the mold carrying seat and the tool identification barcode of the tool holder in the material processing machine, thereby confirming whether or not the mold material and the tool used in the material processing machine is correct;
wherein the mold cleaning module further includes a first water quality detector disposed in the first cleaning tank and a second water quality detector disposed in the second cleaning tank, the first water quality detector is configured to detect a water quality of the liquid medicine in the first cleaning tank, and the second water quality detector is configured to detect a water quality of the clear water in the second cleaning tank.
